(19) United States
(12) Patent Application Publication
(10) Pub. No.: US 2005/0176960 A1
(43) Pub. Date: Aug. 11, 2005

(54) Resolution of racemates of methyl alpha-5-[4,5,6,7-tetrahydro[3,2-C]thienopyridyl]-(2-chlorophenyl) acetate

(75) Inventors: K. S. Keshava Murthy, Ancaster (CA); Elena Bejan, Brantford (CA); Gamini Weeratunga, Brantford (CA)

Correspondence Address: IVOR M. HUGHES, BARRISTER & SOLICITOR,;PATENT & TRADEMARK AGENTS, 175 COMMERCE VALLEY DRIVE WEST, SUITE 200, THORNHILL, ON, L3T 7P6, CA

(73) Assignee: Brantford Chemicals Inc.

(21) Appl. No.: 10/779,724

(22) Filed: Feb. 18, 2004

(30) Foreign Application Priority Data
Feb. 11, 2004 (CA) 2,457,459

(51) Int. Cl. C07D 498/02

(52) U.S. Cl. 546/114

(57) ABSTRACT

A process for the resolution of each of the enantiomers of methyl-α-5-[4,5,6,7-tetrahydro[3,2-c]thienopyridyl]-(2-chlorophenyl)acetate and salts thereof by diastereomeric crystallization comprising the use of a single optically active resolving agent and at least one solvent.

### FIELD OF THE INVENTION

The present invention relates to a novel process for the resolution of mixtures of the compound methyl-α-5-[4,5,6,7-tetrahydro[3,2-c]thienopyridyl]-(2-chlorophenyl)acetate and to a novel salt form of its (S)-enantiomer.

### BACKGROUND OF THE INVENTION

The dextrorotatory or (S)-enantiomer of methyl-α-5-[4,5,6,7-tetrahydro[3,2-c]thienopyridyl]-(2-chlorophenyl)acetate is known generically as Clopidogrel. Clopidogrel is a known inhibitor of ADP-induced platelet aggregation and possesses antithrombotic activities. The levorotatory or (R)-enantiomer of this compound is described in U.S. Pat. No. 5,225,420 as being useful as an angiogenesis inhibitor.

It is known in the art to prepare each of the single enantiomers of methyl-α-5-[4,5,6,7-tetrahydro[3,2-c]thienopyridyl]-(2-chlorophenyl)acetate by means of enantioselective synthesis; see for example U.S. Pat. No. 6,495,691 and the references cited within.

Another process, disclosed in U.S. Pat. No. 4,847,265, for the preparation of each of the (S)- and (R)-enantiomers of methyl-α-5-[4,5,6,7-tetrahydro[3,2-c]thienopyridyl]-(2-chlorophenyl)acetate comprises isolation of each of these enantiomers by crystallization of diastereomeric salts formed with levorotatory ((R)-enantiomer) or dextrorotatory ((S)-enantiomer) 10-camphorsulfonic acid respectively.

For the isolation of the (S)-enantiomer of methyl-α-5-[4,5,6,7-tetrahydro[3,2-c]thienopyridyl]-(2-chlorophenyl)acetate, Clopidogrel, U.S. Pat. No. 4,847,265 teaches reacting a racemic mixture of methyl-α-5-[4,5,6,7-tetrahydro[3,2-c]thienopyridyl]-(2-chlorophenyl)acetate in a solvent, ideally acetone, with (R)-10-camphorsulfonic acid to form a diastereomeric salt, the (S)-methyl-α-5-[4,5,6,7-tetrahydro[3,2-c]thienopyridyl]-(2-chlorophenyl)acetate (R)-10-camphorsulfonic acid salt, followed by repeated recrystallizations of said salt from a solvent such as acetone until a constant optical rotation for the precipitated diastereomeric salt is obtained. The desired (S)-methyl-α-5-[4,5,6,7-tetrahydro[3,2-c]thienopyridyl]-(2-chlorophenyl)acetate enantiomer is then liberated from the diastereomeric salt as the free base by the action of a base such as sodium or potassium hydrogen carbonate in aqueous media.

U.S. Pat. No. 4,847,265 also teaches that the enantiomeric purity of the (S)-enantiomer of methyl-α-5-[4,5,6,7-tetrahydro[3,2-c]thienopyridyl]-(2-chlorophenyl)acetate obtained according to its examples, which included recrystallization of the (S)-methyl-α-5-[4,5,6,7-tetrahydro[3,2-c]thienopyridyl]-(2-chlorophenyl)acetate (R)-10-camphorsulfonic acid salt from acetone, can be as low as 96%.

For the isolation of the (R)-enantiomer of methyl-α-5-[4,5,6,7-tetrahydro[3,2-c]thienopyridyl]-(2-chlorophenyl)acetate, U.S. Pat. No. 4,847,265 teaches reacting a mixture containing an enantiomeric excess of (R)-methyl-α-5-[4,5,6,7-tetrahydro[3,2-c]thienopyridyl]-(2-chlorophenyl)acetate with (S)-10-camphorsulfonic acid in a solvent to form a diastereomeric salt, the (R)-methyl-α-5-[4,5,6,7-tetrahydro[3,2-c]thienopyridyl]-(2-chlorophenyl)acetate (S)-10-camphorsulfonic acid salt, followed by repeated recrystallizations of said salt from acetone until a constant optical rotation value for the precipitated diastereomeric salt is obtained. The desired (R)-methyl-α-5-[4,5,6,7-tetrahydro[3,2-c]thienopyridyl]-(2-chlorophenyl)acetate enantiomer is then liberated from the diastereomeric salt as the free base by the action of a base such as sodium or potassium hydrogen carbonate in aqueous media.

A drawback of the resolution process described in U.S. Pat. No. 4,847,265 is that it requires the use of (R)-10-camphorsulfonic acid as the optically active resolving agent for the resolution and isolation of the more desirable (S)-enantiomer, which is Clopidogrel. (R)-10-Camphorsulfonic acid is a more expensive reagent compared to (S)-10-camphorsulfonic acid. Additionally, the resolution process described in U.S. Pat. No. 4,847,265 requires the use of both the (R)- and the (S)-enantiomers of 10-camphorsulfonic acid for the resolution and isolation of both the (S)- and (R)-enantiomers of methyl-α-5-[4,5,6,7-tetrahydro[3,2-c]thienopyridyl]-(2-chlorophenyl)acetate. Moreover, an enantiomeric purity of 96% for the (S)-enantiomer, Clopidogrel, produced by this prior art resolution process is undesirable since the (R)-enantiomer is treated as an impurity in pharmaceutical preparations containing Clopidogrel, and, therefore, a higher enantiomeric purity is required.

Another shortcoming of the resolution process described in U.S. Pat. No. 4,847,265 is that the preparation of the (R)-enantiomer requires the use of an enriched material that contains an enantiomeric excess of the (R)-enantiomer compared to the (S)-enantiomer.

Also noteworthy is that repeated recrystallizations of both (R)- and (S)-enantiomers of the methyl-α-5-[4,5,6,7-tetrahydro[3,2-c]thienopyridyl]-(2-chlorophenyl)acetate salts are required in order to achieve high enantiomeric purity levels.

U.S. Pat. No. 4,847,265, along with U.S. Pat. No. 4,529,596 and U.S. Pat. No. 5,204,469, are sources of the review article “Clopidogrel Hydrogensulfate”, Drugs of the Future, (1993), 18(2), p. 107-112, which provides a review of processes for formation of dextro clopidogrel using levo camphorsulfonic acid (although the text and flowsheet found in the article mistakenly states the use of dextro camphorsulfonic acid in the resolution of the racemic clopidogrel).

It is therefore an object of the present invention to provide a novel resolution process wherein the more desirable (S)-enantiomer, Clopidogrel, can be resolved and isolated by means of the use of the more economical enantiomer of an optically active resolving agent.

Another object of the present invention is to provide a process that allows the resolution and isolation of the other enantiomer, (R)-methyl-α-5-[4,5,6,7-tetrahydro[3,2-c]thienopyridyl]-(2-chlorophenyl)acetate, by means of the same enantiomer of the optically active resolving agent, thereby eliminating the need for the more expensive enantiomer of the optically active resolving agent altogether.

Another object of the present invention is to provide a process that will produce the (S)-enantiomer, Clopidogrel, with a high level of enantiomeric purity (98%).

Another object of this invention is to provide a process to recycle the mixture obtained after the isolation of pure (S)-methyl-α-5-[4,5,6,7-tetrahydro[3,2-c]thienopyridyl]-(2-chlorophenyl)acetate (S)-10-camphorsulfonic acid salt.

Furthermore, and another object of this invention, is to provide a process for the conversion of the undesired chemically pure (R)-enantiomer to a racemic mixture of (R)- and (S)-enantiomers from which the more desired (S)-enantiomer can be isolated.

The advantages of the current process, such as cost efficiency and simplicity, are the result of the novel choice of the resolving agent and also of the utilization of commercially viable solvent systems, thereby permitting the isolation of both (R)- and (S)-enantiomers of the methyl-α-5-[4,5,6,7-tetrahydro[3,2-c]thienopyridyl]-(2-chlorophenyl)acetate with an excellent enantiomeric purity directly from the reaction mixture without any additional procedures. In the case of a single solvent system, the solvent could be recovered and recycled, therefore increasing the cost efficiency of the process.

Further advantages associated with the present invention will be readily perceived in reviewing the summary of the invention.

Further and other objects of the invention will be apparent to those skilled in the art from the following summary of the invention and the detailed description of embodiments thereof.

### SUMMARY OF THE INVENTION

Unexpectedly, we have found that by using the (S)-10-camphorsulfonic acid as the optically active resolving agent we can obtain both (R)- and (S)-enantiomers of the methyl-α-5-[4,5,6,7-tetrahydro[3,2-c]thienopyridyl]-(2-chlorophenyl)acetate in optically pure form.

The process described in this invention is more advantageous than the prior art as it efficiently provides the (S)-enantiomer of the methyl-α-5-[4,5,6,7-tetrahydro[3,2-c]thienopyridyl]-(2-chlorophenyl)acetate with a high enantiomeric purity (98%) by using the more economical enantiomer of an optically active resolving agent.

Both (R)-methyl-α-5-[4,5,6,7-tetrahydro[3,2-c]thienopyridyl]-(2-chlorophenyl)acetate (S)-10-camphorsulfonic acid and (S)-methyl-α-5-[4,5,6,7-tetrahydro[3,2-c]thienopyridyl]-(2-chlorophenyl)acetate (S)-10-camphorsulfonic acid salts can be recrystallized further in order to prepare materials with enantiomeric purity as high as 99.8%.

Moreover, it has been found that both diastereomeric salts formed with (S)-10-camphorsulfonic acid are crystalline and non-hygroscopic, resulting in advantageous filtration and drying characteristics.

The resolution process according to the invention comprises: reacting a mixture of enantiomers of methyl-α-5-[4,5,6,7-tetrahydro[3,2-c]thienopyridyl]-(2-chlorophenyl)acetate with (S)-10-camphorsulfonic acid; filtering off the (R)-methyl-α-5-[4,5,6,7-tetrahydro[3,2-c]thienopyridyl]-(2-chlorophenyl)acetate (S)-10-camphorsulfonic acid salt which initially forms; adding an additional amount of (S)-10-camphorsulfonic acid; filtering off the (S)-methyl-α-5-[4,5,6,7-tetrahydro[3,2-c]thienopyridyl]-(2-chlorophenyl)acetate (S)-10-camphorsulfonic acid salt which forms; if required, recrystallizing the diastereomeric salts; and, converting the diastereomeric salts to their free base, the purified single enantiomers, in a standard manner, such as by the action of a base such as sodium or potassium hydrogen carbonate in aqueous media.

In the case of a mixture enriched in the undesired enantiomer, it may be preferable to perform an initial racemization step by using a base in an organic solvent as described in the following paragraph.

As an additional step, the (R)-methyl-α-5-[4,5,6,7-tetrahydro[3,2-c]thienopyridyl]-(2-chlorophenyl)acetate (S)-10-camphorsulfonic acid salt isolated first is converted to the free base and racemized in the presence of a base in an organic solvent then recycled through the process along with the filtrate obtained after isolation of the (S)-enantiomer of the methyl-α-5-[4,5,6,7-tetrahydro[3,2-c]thienopyridyl]-(2-chlorophenyl)acetate (S)-10-camphorsulfonic acid salt.

Suitable solvents for the resolution process and recrystallizations according to the invention include C2 to C6 ketones, such as methyl isobutyl ketone, methyl ethyl ketone, and toluene and mixtures thereof. Toluene is particularly preferred as the solvent for both the resolution and recrystallizations. The use of toluene is particularly surprising in light of the fact that the prior art teaches the use of polar solvents. Thus, another aspect of the present invention is the discovery that a non-polar solvent such as toluene can be used as a solvent for the resolution and recrystallization of both (R)-methyl-α-5-[4,5,6,7-tetrahydro[3,2-c]thienopyridyl]-(2-chlorophenyl)acetate (S)-10-camphorsulfonic acid and (S)-methyl-α-5-[4,5,6,7-tetrahydro[3,2-c]thienopyridyl]-(2-chlorophenyl)acetate (S)-10-camphorsulfonic acid salts.

The (S)-methyl-α-5-[4,5,6,7-tetrahydro[3,2-c]thienopyridyl]-(2-chlorophenyl)acetate (S)-10-camphorsulfonic acid salt may be free based and then transformed into Clopidogrel Bisulphate.

According to one aspect of the invention, there is provided a process for the resolution of each of the enantiomers of methyl-α-5-[4,5,6,7-tetrahydro[3,2-c]thienopyridyl]-(2-chlorophenyl)acetate and salts thereof by diastereomeric crystallization comprising the use of a single optically active resolving agent and at least one solvent, preferably the optically active resolving agent is (S)-10-camphorsulfonic acid.

Preferably the solvent is selected from a polar organic solvent, preferably the polar organic solvent is a C2 to C6 ketone.

Even more preferably the polar organic solvent is selected from the group consisting of methyl ethyl ketone and methyl isobutyl ketone.

In another embodiment of the invention, the solvent in the process is preferably a non-polar organic solvent, preferably toluene.

According to another aspect of the invention, the process further comprises recrystallization to an enantiomeric purity of about 99.5% or higher by dissolution in an organic solvent and recrystallization, preferably the organic solvent is selected from the group consisting of toluene, methyl isobutyl ketone, methyl ethyl ketone or a mixture thereof.

According to another aspect of the invention, there is provided a process for the preparation of (S)-methyl-α-5-[4,5,6,7-tetrahydro[3,2-c]thienopyridyl]-(2-chlorophenyl)acetate (S)-10-camphorsulfonic acid salt by diastereomeric crystallization of a mixture of the enantiomers of methyl-α-5-[4,5,6,7-tetrahydro[3,2-c]thienopyridyl]-(2-chlorophenyl)acetate comprising the use of (S)-10-camphorsulfonic acid as the optically active resolving agent in the presence of at least one solvent, preferably the solvent is a polar organic solvent, preferably the polar organic solvent is a C2 to C6 ketone.

Even more preferably the polar organic solvent is selected from the group consisting of methyl ethyl ketone and methyl isobutyl ketone.

In another embodiment of the invention the solvent is a non-polar organic solvent, preferably toluene.

According to another aspect of the invention, there is provided a process for the preparation of (R)-methyl-α-5-[4,5,6,7-tetrahydro[3,2-c]thienopyridyl]-(2-chlorophenyl)acetate (S)-10-camphorsulfonic acid salt by diastereomeric crystallization comprising the use of (S)-10-camphorsulfonic acid as the optically active resolving agent.

According to yet another aspect of the invention, there is provided a process for resolving a diastereomeric mixture containing (S)-methyl-α-5-[4,5,6,7-tetrahydro[3,2-c]thienopyridyl]-(2-chlorophenyl)acetate (S)-10-camphorsulfonic acid salt and (R)-methyl-α-5-[4,5,6,7-tetrahydro[3,2-c]thienopyridyl]-(2-chlorophenyl)acetate (S)-10-camphorsulfonic acid, which comprises dissolving said mixture in a solvent or a solvent mixture and crystallizing (S)-methyl-α-5-[4,5,6,7-tetrahydro[3,2-c]thienopyridyl]-(2-chlorophenyl)acetate (S)-10-camphorsulfonic acid salt, preferably, the solvent is selected from a polar organic solvent, preferably the solvent is a C2 to C6 ketone, even more preferably the solvent is selected from the group consisting of methyl ethyl ketone and methyl isobutyl ketone.

In another instance the solvent is a non-polar organic solvent, preferably toluene.

According to yet another aspect of the invention, there is provided the compound (S)-methyl-α-5-[4,5,6,7-tetrahydro[3,2-c]thienopyridyl]-(2-chlorophenyl)acetate (S)-10-camphorsulfonic acid salt, substantially free of (R)-methyl-α-5-[4,5,6,7-tetrahydro[3,2-c]thienopyridyl]-(2-chlorophenyl)acetate (S)-10-camphorsulfonic acid salt.

According to yet another aspect of the invention, there is provided the compound (S)-methyl-α-5-[4,5,6,7-tetrahydro[3,2-c]thienopyridyl]-(2-chlorophenyl)acetate (S)-10-camphorsulfonic acid salt with an enantiomeric purity of about 98% or more.

According to yet another aspect of the invention, there is provided the compound (S)-methyl-α-5-[4,5,6,7-tetrahydro[3,2-c]thienopyridyl]-(2-chlorophenyl)acetate hydrogen sulfate salt with an enantiomeric purity of about 98% or more, prepared by free basing (S)-methyl-α-5-[4,5,6,7-tetrahydro[3,2-c]thienopyridyl]-(2-chlorophenyl)acetate (S)-10-camphorsulfonic acid salt and further transformation to the hydrogen sulphate salt.

According to yet another aspect of the invention, any of the processes may further comprise the addition of seeds of the product.

### EXAMPLES

The following examples serve to illustrate embodiments of the present invention in a manner in which they can be practiced but, as such, should not be considered in a limiting sense.

### Example 1

a) (R)-methyl-α-5-[4,5,6,7-tetrahydro[3,2-c]thienopyridyl]-(2-chlorophenyl)acetate (S)-10-camphorsulfonic acid salt

Racemic methyl-α-5-[4,5,6,7-tetrahydro[3,2-c]thienopyridyl]-(2-chlorophenyl)acetate (200 g) was added of 1200 mL of toluene and treated with 57.75 g (1S)-(+)-10-camphorsulfonic acid. The solution was stirred at room temperature for 15 minutes. (R)-Methyl-α-5-[4,5,6,7-tetrahydro[3,2-c]thienopyridyl]-(2-chlorophenyl)acetate (S)-10-camphorsulfonic acid (2.0 g) was added and stirring continued for 5 hours at room temperature. The reaction mixture was filtered and washed with 100 mL of toluene. After drying, 110.21 g of (R)-methyl-α-5-[4,5,6,7-tetrahydro[3,2-c]thienopyridyl]-(2-chlorophenyl)acetate (S)-10-camphorsulfonic acid salt was obtained. (yield: 32%; enantiomeric purity by chiral HPLC: 90.88%)

b) (S)-methyl-α-5-[4,5,6,7-tetrahydro[3,2-c]thienopyridyl]-(2-chlorophenyl)acetate (S)-10-camphorsulfonic acid salt

After separation of the (R)-methyl-α-5-[4,5,6,7-tetrahydro[3,2-c]thienopyridyl]-(2-chlorophenyl)acetate (S)-10-camphorsulfonic acid salt in Example 1 a), half of the mother liquor was evaporated to 467 mL and then treated with 43.31 g (1S)-(+)-10-camphorsulfonic acid. The reaction mixture was heated to reflux temperature, cooled to 32-35° C. and then (S)-methyl-α-5-[4,5,6,7-tetrahydro[3,2-c]thienopyridyl]-(2-chlorophenyl)acetate (S)-10-camphorsulfonic acid (1.1 g) was added. Stirring was continued for 2 hours at room temperature. The reaction mixture was filtered and washed with 100 mL of toluene. After drying, 48.88 g of (S)-methyl-α-5-[4,5,6,7-tetrahydro[3,2-c]thienopyridyl]-(2-chlorophenyl)acetate (S)-10-camphorsulfonic acid salt was obtained. (yield: 41.8%; enantiomeric purity by chiral HPLC: 98.69%). A sample from the filtrate was evaporated to dryness, dissolved in methanol then analyzed by chiral HPLC. (enantiomeric ratio: 58.15% S/41.85% R)

### Example 2

a) (R)-methyl-α-5-[4,5,6,7-tetrahydro[3,2-c]thienopyridyl]-(2-chlorophenyl)acetate (S)-10-camphorsulfonic acid salt

Racemic methyl-α-5-[4,5,6,7-tetrahydro[3,2-c]thienopyridyl]-(2-chlorophenyl)acetate (100.1 g) in 600 mL of toluene and 11 mL methyl isobutyl ketone was treated with 28.9 g (1S)-(+)-10-camphorsulfonic acid. The solution was stirred at room temperature for 15 minutes. (R)-Methyl-α-5-[4,5,6,7-tetrahydro[3,2-c]thienopyridyl]-(2-chlorophenyl)acetate (S)-10-camphorsulfonic acid (1.0 g) was added and stirring was continued for 5 hours at room temperature. The reaction mixture was filtered and washed with 25 mL of toluene. After drying, 52.11 g of (R)-methyl-α-5-[4,5,6,7-tetrahydro[3,2-c]thienopyridyl]-(2-chlorophenyl)acetate (S)-10-camphorsulfonic acid salt was obtained. (yield: 30.2%; enantiomeric purity by chiral HPLC: 92.01%)

b) (S)-methyl-α-5-[4,5,6,7-tetrahydro[3,2-c]thienopyridyl]-(2-chlorophenyl)acetate (S)-10-camphorsulfonic acid salt

After separation of the (R)-methyl-α-5-[4,5,6,7-tetrahydro[3,2-c]thienopyridyl]-(2-chlorophenyl)acetate (S)-10-camphorsulfonic acid salt in Example 2 a), half of the mother liquor was evaporated to 120 mL and 180 mL methyl ethyl ketone was added followed by 21.6 g (1S)-(+)-10-camphorsulfonic acid. The reaction mixture was heated to reflux temperature till a clear solution was obtained. After cooling to 32-35° C., (S)-methyl-α-5-[4,5,6,7-tetrahydro[3,2-c]thienopyridyl]-(2-chlorophenyl)acetate (S)-10-camphorsulfonic acid (1.2 g) was added and stirring was continued for 2 hours at room temperature. The reaction mixture was filtered and washed with 60 mL of methyl ethyl ketone. After drying, 14.6 g of (S)-methyl-α-5-[4,5,6,7-tetrahydro[3,2-c]thienopyridyl]-(2-chlorophenyl)acetate (S)-10-camphorsulfonic acid salt was obtained. (yield: 24.2%; enantiomeric purity by chiral HPLC: 99.76%)

¹H NMR (DMSO-d6, ppm) 7.68-7.62 (2H, m); 7.52-7.50 (2H, m); 7.41 (d, ³J_{H—H}=4.9 Hz); 6.86 (d, ³J_{H—H}=4.9 Hz); 5.48 (1H, bs); 4.3-3.8 (2H; bs); 3.74 (3H, bs); 3.6-3.2 (2H, bs); 3.1-2.9 (2H, bs); 2.85 (1H, d, J=14.7 Hz); 2.7-2.6 (1H, m); 2.37 (1H, J=14.7 Hz); 2.28-2.19 (1H, m); 1.95-1.76 (3H, m); 1.32-1.22 (2H, m); 1.04 (3H, s); 0.74 (3H, s).

### Example 3

a) (R)-methyl-α-5-[4,5,6,7-tetrahydro[3,2-c]thienopyridyl]-(2-chlorophenyl)acetate (S)-10-camphorsulfonic acid salt

Racemic methyl-α-5-[4,5,6,7-tetrahydro[3,2-c]thienopyridyl]-(2-chlorophenyl)acetate (100 g) was added of 400 mL of methyl isobutyl ketone and treated with 28.87 g (1S)-(+)-10-camphorsulfonic acid. The solution was stirred at room temperature and then (R)-methyl-α-5-[4,5,6,7-tetrahydro[3,2-c]thienopyridyl]-(2-chlorophenyl)acetate (S)-10-camphorsulfonic acid (1.0 g) was added and stirring was continued for 5 hours at room temperature. The reaction mixture was filtered and washed with 100 mL of methyl isobutyl ketone. After drying, 50.87 g of (R)-methyl-α-5-[4,5,6,7-tetrahydro[3,2-c]thienopyridyl]-(2-chlorophenyl)acetate (S)-10-camphorsulfonic acid salt was obtained. (yield: 29.6%; enantiomeric purity by chiral HPLC: 95.64%)

b) (S)-methyl-α-5-[4,5,6,7-tetrahydro[3,2-c]thienopyridyl]-(2-chlorophenyl)acetate (S)-10-camphorsulfonic acid salt

After separation of the (R)-methyl-α-5-[4,5,6,7-tetrahydro[3,2-c]thienopyridyl]-(2-chlorophenyl)acetate (S)-10-camphorsulfonic acid salt in Example 3 a), one portion of the mother liquor (one tenth) was evaporated to 24 mL, 24 mL of toluene was added followed by 5.15 g (1S)-(+)-10-camphorsulfonic acid. After stirring at room temperature, (S)-methyl-α-5-[4,5,6,7-tetrahydro[3,2-c]thienopyridyl]-(2-chlorophenyl)acetate (S)-10-camphorsulfonic acid (0.12 g) was added and stirring was continued for 18 hours at room temperature. The reaction mixture was then heated to reflux temperature till a clear solution was obtained. After cooling, the reaction mixture was stirred for 18 hours at room temperature. The suspension was filtered and washed with 60 mL of toluene. After drying, 3.62 g of (S)-methyl-α-5-[4,5,6,7-tetrahydro[3,2-c]thienopyridyl]-(2-chlorophenyl)acetate (S)-10-camphorsulfonic acid salt was obtained. (yield: 29.9%; enantiomeric purity by chiral HPLC: 98.77%)

### Example 4

a) (R)-methyl-α-5-[4,5,6,7-tetrahydro[3,2-c]thienopyridyl]-(2-chlorophenyl)acetate (S)-10-camphorsulfonic acid salt

Racemic methyl-α-5-[4,5,6,7-tetrahydro[3,2-c]thienopyridyl]-(2-chlorophenyl)acetate (100 g) was added of 400 mL of methyl isobutyl ketone and further treated with 28.87 g (1S)-(+)-10-camphorsulfonic acid. The solution was stirred at room temperature for 15 minutes. (R)-Methyl-α-5-[4,5,6,7-tetrahydro[3,2-c]thienopyridyl]-(2-chlorophenyl)acetate (S)-10-camphorsulfonic acid (1.0 g) was added and stirring was continued for 5 hours at room temperature. The reaction mixture was filtered and washed with 100 mL of methyl isobutyl ketone. After drying, 50.87 g of (R)-methyl-α-5-[4,5,6,7-tetrahydro[3,2-c]thienopyridyl]-(2-chlorophenyl)acetate (S)-10-camphorsulfonic acid salt was obtained. (yield: 29.6%; enantiomeric purity by chiral HPLC: 95.64%)

b) (S)-methyl-α-5-[4,5,6,7-tetrahydro[3,2-c]thienopyridyl]-(2-chlorophenyl)acetate (S)-10-camphorsulfonic acid salt

After separation of the (R)-methyl-α-5-[4,5,6,7-tetrahydro[3,2-c]thienopyridyl]-(2-chlorophenyl)acetate (S)-10-camphorsulfonic acid salt in Example 4 a), one portion of the mother liquor (one tenth) was treated with 5.15 g of (1S)-(+)-10-camphorsulfonic acid. After 15 minutes of stirring at room temperature, the reaction mixture was combined with 42 mL methyl isobutyl ketone and (S)-methyl-α-5-[4,5,6,7-tetrahydro[3,2-c]thienopyridyl]-(2-chlorophenyl)acetate (S)-10-camphorsulfonic acid (0.12 g) was added. Stirring was continued for 18 hours at room temperature. The suspension was then filtered and washed with 6 mL of methyl isobutyl ketone. After drying, 4.27 g of (S)-methyl-α-5-[4,5,6,7-tetrahydro[3,2-c]thienopyridyl]-(2-chlorophenyl)acetate (S)-10-camphorsulfonic acid salt was obtained. (yield: 35.3%; enantiomeric purity by chiral HPLC: 98.88%)

### Example 5

a) (R)-methyl-α-5-[4,5,6,7-tetrahydro[3,2-c]thienopyridyl]-(2-chlorophenyl)acetate (S)-10-camphorsulfonic acid salt

Racemic methyl-α-5-[4,5,6,7-tetrahydro[3,2-c]thienopyridyl]-(2-chlorophenyl)acetate (27.16 g) in 163 ml toluene was treated with 9.8 g of (1S)-(+)-10-camphorsulfonic acid. The mixture was stirred at room temperature until a solution was obtained. (R)-Methyl-α-5-[4,5,6,7-tetrahydro[3,2-c]thienopyridyl]-(2-chlorophenyl)acetate (S)-10-camphorsulfonic acid salt (0.27 g) was added and stirring was continued for 5 hours at room temperature. The reaction mixture was filtered and washed with 13 mL of toluene. After drying, 13.97 g of (R)-methyl-α-5-[4,5,6,7-tetrahydro[3,2-c]thienopyridyl]-(2-chlorophenyl)acetate (S)-10-camphorsulfonic acid salt was obtained. (yield: 29.9%; enantiomeric purity by chiral HPLC: 97.07%)

b) (S)-methyl-α-5-[4,5,6,7-tetrahydro[3,2-c]thienopyridyl]-(2-chlorophenyl)acetate (S)-10-camphorsulfonic acid salt

After separation of the (R)-methyl-α-5-[4,5,6,7-tetrahydro[3,2-c]thienopyridyl]-(2-chlorophenyl)acetate (S)-10-camphorsulfonic acid salt in Example 5 a), the mother liquor was evaporated to 131 mL and then treated with 9.8 g of (1S)-(+)-10-camphorsulfonic acid. After 5 minutes of stirring at room temperature, the reaction mixture was heated at reflux temperature till a solution was obtained. After cooling to 32-35° C., (S)-methyl-α-5-[4,5,6,7-tetrahydro[3,2-c]thienopyridyl]-(2-chlorophenyl)acetate (S)-10-camphorsulfonic acid (0.32 g) was added and stirring was continued for 2 hours at room temperature. The reaction mixture was filtered and washed with 33 mL of toluene. After drying, 10.69 g of (S)-methyl-α-5-[4,5,6,7-tetrahydro[3,2-c]thienopyridyl]-(2-chlorophenyl)acetate (S)-10-camphorsulfonic acid salt was obtained. (yield: 40.3%; enantiomeric purity by chiral HPLC: 98.82%)

### Example 6

a) (R)-methyl-α-5-[4,5,6,7-tetrahydro[3,2-c]thienopyridyl]-(2-chlorophenyl)acetate (S)-10-camphorsulfonic acid salt

Racemic methyl-α-5-[4,5,6,7-tetrahydro[3,2-c]thienopyridyl]-(2-chlorophenyl)acetate (25 g) in 175 ml toluene was treated with 10.83 g (1S)-(+)-10-camphorsulfonic acid. The solution was stirred at room temperature and then (R)-methyl-α-5-[4,5,6,7-tetrahydro[3,2-c]thienopyridyl]-(2-chlorophenyl)acetate (S)-10-camphorsulfonic acid salt (0.25 g) was added. Stirring was continued for 5 hours at room temperature. The reaction mixture was filtered and washed with 13 mL of toluene. After drying, 15.86 g of (R)-methyl-α-5-[4,5,6,7-tetrahydro[3,2-c]thienopyridyl]-(2-chlorophenyl)acetate (S)-10-camphorsulfonic acid salt were obtained. (yield: 36.8%; enantiomeric purity by chiral HPLC: 96.71%)

b) (S)-methyl-α-5-[4,5,6,7-tetrahydro[3,2-c]thienopyridyl]-(2-chlorophenyl)acetate (S)-10-camphorsulfonic acid salt

After separation of the (R)-methyl-α-5-[4,5,6,7-tetrahydro[3,2-c]thienopyridyl]-(2-chlorophenyl)acetate (S)-10-camphorsulfonic acid salt in Example 6 a), the mother liquor was evaporated to 108 mL and then treated with 7.2 g (1S)-(+)-10-camphorsulfonic acid. After stirring at room temperature, the reaction mixture was heated at reflux temperature till a solution was obtained. After cooling to 32-35° C., (S)-methyl-α-5-[4,5,6,7-tetrahydro[3,2-c]thienopyridyl]-(2-chlorophenyl)acetate (S)-10-camphorsulfonic acid (0.27 g) was added and stirring was continued for 2 hours at room temperature.

The reaction mixture was filtered and washed with 27 mL of toluene. After drying, 10.95 g of (S)-methyl-α-5-[4,5,6,7-tetrahydro[3,2-c]thienopyridyl]-(2-chlorophenyl)acetate (S)-10-camphorsulfonic acid salt was obtained. (yield: 40.3%; enantiomeric purity by chiral HPLC: 98.82%)

### Example 7

Recrystallization of (S)-methyl-α-5-[4,5,6,7-tetrahydro[3,2-c]thienopyridyl]-(2-chlorophenyl)acetate (S)-10-camphorsulfonic acid salt

(S)-methyl-α-5-[4,5,6,7-tetrahydro[3,2-c]thienopyridyl]-(2-chlorophenyl)acetate (S)-10-camphorsulfonic acid salt (3 g), prepared as describe at Examples 1-6 b), was added of 9 mL of methyl ethyl ketone and heated to reflux temperature till a solution was obtained. After cooling and stirring at room temperature for 2 hours, the reaction mixture was filtered and washed with 2 mL of methyl ethyl ketone. After drying, 2.16 g of (S)-methyl-α-5-[4,5,6,7-tetrahydro[3,2-c]thienopyridyl]-(2-chlorophenyl)acetate (S)-10-camphorsulfonic acid salt was obtained. (yield: 72%; enantiomeric purity by chiral HPLC: 99.75%)

### Example 8

Recrystallization of (S)-methyl-α-5-[4,5,6,7-tetrahydro[3,2-c]thienopyridyl]-(2-chlorophenyl)acetate (S)-10-camphorsulfonic acid salt

(S)-methyl-α-5-[4,5,6,7-tetrahydro[3,2-c]thienopyridyl]-(2-chlorophenyl)acetate (S)-10-camphorsulfonic acid salt (3 g) prepared as describe at Examples 1-6 b) was suspended in 6 mL of toluene and heated to reflux temperature till a solution was obtained. After cooling and stirring at room temperature for 4 hours, the reaction mixture was filtered and washed with 6 mL of toluene. After drying, 2.45 g of (S)-methyl-α-5-[4,5,6,7-tetrahydro[3,2-c]thienopyridyl]-(2-chlorophenyl)acetate (S)-10-camphorsulfonic acid salt was obtained. (yield: 82%; enantiomeric purity by chiral HPLC: 99.5%)

### Example 9

Recrystallization of (R)-methyl-α-5-[4,5,6,7-tetrahydro[3,2-c]thienopyridyl]-(2-chlorophenyl)acetate (S)-10-camphorsulfonic acid salt

(R)-methyl-α-5-[4,5,6,7-tetrahydro[3,2-c]thienopyridyl]-(2-chlorophenyl)acetate (S)-10-camphorsulfonic acid salt (69.09 g), prepared as describe at Examples 1-6 a), was suspended in 898 mL of acetone and 27.84 mL of methanol and heated to reflux temperature till a solution was obtained. After slowly cooling to room temperature, a suspension was formed which was further cooled to 0-5° C. and stirred for 3 hours. The reaction mixture was filtered and washed with 70 mL of cold acetone. After drying, 50.28 g of (R)-methyl-α-5-[4,5,6,7-tetrahydro[3,2-c]thienopyridyl]-(2-chlorophenyl)acetate (S)-10-camphorsulfonic acid salt was obtained. (yield: 73%; enantiomeric purity by chiral HPLC: 99.8%)

### Example 10

a) (R)-methyl-α-5-[4,5,6,7-tetrahydro[3,2-c]thienopyridyl]-(2-chlorophenyl)acetate (S)-10-camphorsulfonic acid salt

Racemic methyl-α-5-[4,5,6,7-tetrahydro[3,2-c]thienopyridyl]-(2-chlorophenyl)acetate (10 g) was added of 80 mL of toluene and treated with 2.89 g (1S)-(+)-10-camphorsulfonic acid. The reaction mixture was stirred at room temperature for 5 hours then filtered and washed with 10 mL of toluene. After drying, 5.79 g of (R)-methyl-α-5-[4,5,6,7-tetrahydro[3,2-c]thienopyridyl]-(2-chlorophenyl)acetate (S)-10-camphorsulfonic acid salt was obtained. (yield: 34%; enantiomeric purity by chiral HPLC: 91.2%)

b) (S)-methyl-α-5-[4,5,6,7-tetrahydro[3,2-c]thienopyridyl]-(2-chlorophenyl)acetate (S)-10-camphorsulfonic acid salt

After separation of the (R)-methyl-α-5-[4,5,6,7-tetrahydro[3,2-c]thienopyridyl]-(2-chlorophenyl)acetate (S)-10-camphorsulfonic acid salt, the mother liquor (67.2 g containing an expected 10 g of (S)-methyl-α-5-[4,5,6,7-tetrahydro[3,2-c]thienopyridyl]-(2-chlorophenyl)acetate (S)-10-camphorsulfonic acid salt) was evaporated to 20 mL and added of 40 mL methyl ethyl ketone. After a Celite™ filtration the reaction mixture was treated with 3.8 g (1S)-(+)-10-camphorsulfonic acid.

The reaction mixture was then heated to reflux temperature, filtered through Celite™ and cooled to room temperature. After stirring for 3 hours at room temperature, the suspension was filtered and washed with 10 mL of toluene. After drying, 4.0 g of (S)-methyl-α-5-[4,5,6,7-tetrahydro[3,2-c]thienopyridyl]-(2-chlorophenyl)acetate (S)-10-camphorsulfonic acid salt was obtained. (yield: 40%; enantiomeric purity by chiral HPLC: 98.5%)

### Example 11

Racemic methyl-α-5-[4,5,6,7-tetrahydro[3,2-c]thienopyridyl]-(2-chlorophenyl)acetate (S)-10-camphorsulfonic acid

a) (R)-methyl-α-5-[4,5,6,7-tetrahydro[3,2-c]thienopyridyl]-(2-chlorophenyl)acetate (S)-10-camphorsulfonic acid salt (32.4 g) in 260 mL of toluene was stirred with a solution of 6.2 g sodium bicarbonate in 150 mL water. After usual work-up and azeotropic removal of water, the organic layer was heated to 80° C. and sodium methoxide (1.08 g) was added. After cooling to room temperature, the reaction mixture was analyzed for the enantiomeric ratio by chiral HPLC (enantiomeric ratio by chiral HPLC: 50% S/50% R).

b) The filtrate obtained after isolation of the (S)-enantiomer of the methyl-α-5-[4,5,6,7-tetrahydro[3,2-c]thienopyridyl]-(2-chlorophenyl)acetate salt as described in Examples 1-6 b) can be converted to the free base as previously described and then recycled through the process along with the solution obtained in part a).

While the foregoing provides a detailed description of preferred embodiments of the invention, it is to be understood that this description is illustrative only of the principles of the invention and not limitative. Furthermore, as many changes can be made to the embodiments without departing from the scope of the invention, it is intended that all material contained herein be interpreted as illustrative of the invention and not in a limiting sense.

1. A process for the resolution of each of the enantiomers of methyl-α-5-[4,5,6,7-tetrahydro[3,2-c]thienopyridyl]-(2-chlorophenyl)acetate and salts thereof by diastereomeric crystallization comprising the use of a single optically active resolving agent and at least one solvent.

2. A process according to claim 1 wherein the optically active resolving agent is (S)-10-camphorsulfonic acid.

3. A process according to claim 1 wherein the solvent is selected from a polar organic solvent.

4. The process of claim 3 wherein the polar organic solvent is a C2 to C6 ketone.

5. The process of claim 4 wherein the polar organic solvent is selected from the group consisting of methyl ethyl ketone and methyl isobutyl ketone.

6. A process according to claim 1 wherein the solvent is a non-polar organic solvent.

7. A process according to claim 6 wherein the non-polar solvent is toluene.

8. A process according to claim 1 further comprising recrystallization to an enantiomeric purity of about 99.5% or higher by dissolution in an organic solvent and recrystallization.

9. A process according to claim 8 wherein the organic solvent is selected from the group consisting of toluene, methyl isobutyl ketone, methyl ethyl ketone or a mixture thereof.

10. A process for the preparation of (S)-methyl-α-5-[4,5,6,7-tetrahydro[3,2-c]thienopyridyl]-(2-chlorophenyl)acetate (S)-10-camphorsulfonic acid salt by diastereomeric crystallization of a mixture of the enantiomers of methyl-α-5-[4,5,6,7-tetrahydro[3,2-c]thienopyridyl]-(2-chlorophenyl)acetate comprising the use of (S)-10-camphorsulfonic acid as the optically active resolving agent in the presence of at least one solvent.

11. A process according to claim 10 wherein the solvent is a polar organic solvent.

12. The process of claim 11 wherein the polar organic solvent is a C2 to C6 ketone.

13. The process of claim 12 wherein the polar organic solvent is selected from the group consisting of methyl ethyl ketone and methyl isobutyl ketone.

14. A process according to claim 10 wherein the solvent is a non-polar organic solvent.

15. A process according to claims 14 wherein the non-polar organic solvent is toluene.

16. A process for the preparation of (R)-methyl-α-5-[4,5,6,7-tetrahydro[3,2-c]thienopyridyl]-(2-chlorophenyl)acetate (S)-10-camphorsulfonic acid salt by diastereomeric crystallization of a racemic mixture of methyl-α-5-[4,5,6,7-tetrahydro[3,2-c]thienopyridyl]-(2-chlorophenyl)acetate comprising the use of (S)-10-camphorsulfonic acid as the optically active resolving agent.

17. A process for resolving a diastereomeric mixture containing (S)-methyl-α-5-[4,5,6,7-tetrahydro[3,2-c]thienopyridyl]-(2-chlorophenyl)acetate (S)-10-camphorsulfonic acid salt and (R)-methyl-α-5-[4,5,6,7-tetrahydro[3,2-c]thienopyridyl]-(2-chlorophenyl)acetate (S)-10-camphorsulfonic acid salt, which comprises dissolving said mixture in a solvent or a solvent mixture and crystallizing (S)-methyl-α-5-[4,5,6,7-tetrahydro[3,2-c]thienopyridyl]-(2-chlorophenyl)acetate (S)-10-camphorsulfonic acid salt.

18. A process according to claim 17 wherein the solvent is selected from a polar organic solvent.

19. A process according to claim 18 wherein the solvent is a C2 to C6 ketone.

20. A process according to claim 19 wherein the solvent is selected from the group consisting of methyl ethyl ketone and methyl isobutyl ketone.

21. A process according to claim 17 wherein the solvent is a non-polar organic solvent.

22. A process according to claims 21 wherein the solvent is toluene.

23. The compound (S)-methyl-α-5-[4,5,6,7-tetrahydro[3,2-c]thienopyridyl]-(2-chlorophenyl)acetate (S)-10-camphorsulfonic acid salt, substantially free of (R)-methyl-α-5-[4,5,6,7-tetrahydro[3,2-c]thienopyridyl]-(2-chlorophenyl)acetate (S)-10-camphorsulfonic acid salt.

24. The compound (S)-methyl-α-5-[4,5,6,7-tetrahydro[3,2-c]thienopyridyl]-(2-chlorophenyl)acetate (S)-10-camphorsulfonic acid salt with an enantiomeric purity of about 98% or more.

25. The compound (S)-methyl-α-5-[4,5,6,7-tetrahydro[3,2-c]thienopyridyl]-(2-chlorophenyl)acetate hydrogen sulfate salt with an enantiomeric purity of about 98% or more, prepared by free basing the compound of claim 24 and further transformation into the hydrogen sulfate salt.

26. A process according to any one of claims 1 to 22 further comprising the addition of seeds of the product.

27. The compound of claim 24 wherein prepared by any of the processes of claims 1 to 15 and 17 to 22.

28. The compound of claim 25 wherein prepared by any of the processes of claims 1 to 15 and 17 to 22.

29. A process according to any one of claims 1, 10, 16 or 17 wherein a mixture enriched in (R)-methyl-α-5-[4,5,6,7-tetrahydro[3,2-c]thienopyridyl]-(2-chlorophenyl)acetate (S)-10-camphorsulfonic acid salt is racemized to a racemic mixture of clopidogrel free base.

30. The racemization of claim 29, wherein said racemization is carried out in the presence of a base.

31. The racemization of claim 30, wherein said racemization is further carried out in an organic solvent.

32. The racemization of (R)-methyl-α-5-[4,5,6,7-tetrahydro[3,2-c]thienopyridyl]-(2-chlorophenyl)acetate (S)-10-camphorsulfonic acid salt to a racemic mixture.
